(19) United States
(12) Patent Application Publication
(10) Pub. No.: US 2026/0315752 A1
(43) Pub. Date: Oct. 15, 2026

(54) SYSTEMS AND METHODS FOR THROMBUS MITIGATION FIELD OF INVENTION

(71) Applicant: ABIOMED, Inc., Danvers, MA (US)

(72) Inventors: Anastasia Carr, Danvers, MA (US); Isabel Gurijala, Danvers, MA (US); Joseph Mayo, Danvers, MA (US); Dalia Leibowitz, Danvers, MA (US)

(73) Assignee: ABIOMED, Inc., Danvers, MA (US)

(21) Appl. No.: 19/647,290

(22) Filed: Apr. 14, 2026

Related U.S. Application Data
(60) Provisional application No. 63/788,568, filed on Apr. 14, 2025.

(51) Int. Cl. A61M 25/06; A61M 25/00; A61M 25/02; A61M 39/06; A61M 60/865

(52) U.S. Cl. CPC A61M 25/0662; A61M 25/0097; A61M 25/02; A61M 39/06; A61M 60/865; A61M 2039/062; A61M 2039/0626; A61M 2039/064; A61M 2209/04; A61M 2210/12

(57) ABSTRACT

The present disclosure provides various techniques for managing thrombus formation, via access sheath design or via a patency device capable of being inserted into a sheath along with the catheter of a medical device. For example, a dual-slit single-access sheath may include a tubular body with a hub body operably coupled to a proximal end. The hub body may contain a hemostatic valve with two helical slits extending in an axial direction, and a foam pad with two openings centered over the helical slits, allowing for simultaneous insertion of multiple devices through a single access point.

### CROSS-REFERENCE TO RELATED APPLICATIONS

The present application claims priority to U.S. Provisional Patent Application No. 63/788,568, filed Apr. 14, 2025, which is herein incorporated by reference in its entirety.

### TECHNICAL FIELD

The present disclosure relates to intravascular medical devices, and more particularly to intravascular sheaths used for percutaneous procedures.

### BACKGROUND

Intravascular medical procedures have become increasingly common for diagnosing and treating various cardiovascular conditions. These procedures often involve the use of intravascular sheaths to provide access to the patient's vasculature for introducing catheters, guidewires, and other medical devices. Intravascular sheaths typically consist of a hollow tube that is inserted into a blood vessel, allowing for the passage of instruments while maintaining hemostasis.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a single-access sheath may include a tubular body defining a lumen extending from a proximal end to a distal end. The sheath may include a hub body operably coupled to a proximal end of the tubular body. The hub body may define an internal volume of space operably coupled to the lumen. The sheath may include a hemostatic valve disposed within the internal volume of space. The hemostatic valve may include one or two helical slits extending in an axial direction from a proximal surface towards a distal surface. The sheath may include a foam pad disposed within the internal volume of space, proximal to the hemostatic valve, the foam pad defining one or two openings centered over the two helical slits. The number of openings is preferably the same as the number of helical slits.

According to other aspects of the present disclosure, the single-access sheath may include one or more of the following features. The hemostatic valve may be composed of a silicone polymer. The two openings may be identical. The two openings may not be identical. The sheath may include a cap disposed over the foam pad. The hub body may define a side port operably coupled to the internal volume of space. The sheath may include a suture pad coupled to a distal end of the hub body. A cross-sectional shape of each helical slit may define six symmetrical arms extending from a central point, each arm rotated 60 degrees from its adjacent arms. A distal end of the tubular body may be blunted. The two openings may have a circular cross-section. The two openings may have a non-circular cross-section. A center-to-center spacing of the two openings may be 3 mm to 7 mm. A center-to-center spacing of the two openings may be 4 mm to 5 mm. The foam pad may be free of additional guiding holes.

According to another aspect of the present disclosure, a system may include a single-access sheath as disclosed herein and a medical device extending through a first helical slit of the single-access sheath.

According to other aspects of the present disclosure, the system may include one or more of the following features. The system may include a patency device extending through the first helical slit of the single-access sheath. The system may include a patency device extending through a second helical slit of the single-access sheath. The system may include a percutaneous coronary intervention (PCI) device extending through a first helical slit of the single-access sheath. The system may include a percutaneous coronary intervention (PCI) device extending through a second helical slit of the single-access sheath. The system may include a guide wire extending through the first helical slit of the single-access sheath. The system may include a guide wire extending through the second helical slit of the single-access sheath.

According to another aspect of the present disclosure, a method may include introducing a dual-slit single-access sheath as disclosed herein to a patient via an access point. The method may include inserting a guide wire through a first helical slit of the single-access sheath and positioning the guide wire within a blood vessel. The method may include inserting a medical device over the guide wire, through the first helical slit, and into a desired position within the blood vessel. The method may include removing the guide wire.

According to other aspects of the present disclosure, the method may include one or more of the following features. The method may include introducing a second guide wire through a second helical slit of the single-access sheath. The method may include introducing a patency device through a second helical slit of the single-access sheath. The method may include introducing a percutaneous coronary intervention (PCI) device through a second helical slit of the single-access sheath. The access point may be a left femoral artery or a right femoral artery.

According to another aspect of the present disclosure, a patency device for managing thrombus formation may include a flexible split ring having a split where a first end at least partially overlaps a second end, the split ring having a central opening extending from a first surface to a second surface, the central opening configured to be disposed around a catheter of a first medical device. The patency device may include one or more additional openings extending from the first surface to the second surface, each additional opening offset from the central opening. The patency device may include an elongated body having at least one prong at a first end, each prong extending away from the elongated body in an axial direction, each prong configured to extend through one of the additional openings, the elongated body being configured to be disposed between the catheter of the first medical device and a sheath of a second medical device. The patency device may include a handle coupled to the elongated body at a second end.

According to other aspects of the present disclosure, the patency device may include one or more of the following features. The elongate body may define a channel extending from a proximal end to a distal end, the channel configured to slideably receive the catheter of the first medical device. The elongate body may have a partial annulus cross-sectional geometry, the partial annulus having a central angle no more than 180 degrees. The flexible split ring may be comprised of a first polymeric material different from a second polymeric material forming at least part of the elongated body. The flexible split ring and the elongated body may be comprised of a first polymeric material. The flexible split ring may be comprised of a polymeric material and a metal. The metal may include nitinol, stainless steel, cobalt-chromium alloy, platinum, titanium, tantalum, or a combination thereof. The central opening may have a diameter of less than 5 mm. The central opening may have a diameter of less than 3 mm. An outer diameter of the flexible split ring may be less than 10 mm. An outer diameter of the flexible split ring may be less than 5 mm. The handle may define a U-shaped channel, perpendicular to the axial direction of the elongated body, the U-shaped channel being configured to be disposed at least partially around the catheter of the first medical device.

According to another aspect of the present disclosure, a kit for thrombus management may include a patency device as disclosed herein and an access or introducer sheath, where the patency device may be configured to be disposed at least partially within a lumen of the access or introducer sheath.

According to another aspect of the present disclosure, a system may include an access or introducer sheath having a lumen extending from a proximal end to a distal end. The system may include a patency device as disclosed herein, where the flexible split ring may be in contact with a distal end of the access or introducer sheath and the elongated body extends through the access or introducer sheath. The system may include a medical device having a catheter, the catheter disposed through the central opening of the flexible split ring, where the elongated body may be disposed between the catheter and an inner surface of the lumen.

According to other aspects of the present disclosure, the system may include one or more of the following features. The medical device may be an intravascular blood pump.

According to another aspect of the present disclosure, a method may include introducing a medical device through a lumen of an access or introducer sheath, the medical device having a catheter. The method may include passing a portion of catheter through the slit in a patency device as disclosed herein such that the catheter may be disposed through the central opening of the patency device. The method may include moving the flexible split ring distally until the flexible split ring passes beyond a distal end of the access or introducer sheath, then moving the flexible split ring proximally until a proximal surface of the flexible split ring interacts with the access or introducer sheath and forms a seal.

According to other aspects of the present disclosure, the method may include one or more of the following features. Moving the flexible split ring proximally may include causing a portion of the flexible split ring to be disposed within the lumen. The seal may be a 360 degree seal around the catheter. The seal may be less than a 360 degree seal around the catheter. The access or introducer sheath may be a dual-slit single-access sheath as disclosed herein.

According to another aspect of the present disclosure, a patency device for managing thrombus formation may include an elongated body having a proximal end and a distal end, and extending in an axial direction. The patency device may include an occlusive portion at the distal end of the elongated body, the occlusive portion having a distal end, an intermediate portion with a larger diameter than a diameter of the elongated body, and a tapered proximal end, the occlusive portion defining a c-shaped channel extending in an axial direction, the c-shaped channel configured to wrap partially around a catheter of a medical device, the occlusive portion configured to be disposed between the catheter and a distal end of a lumen of an access sheath. The patency device may include a handle having a u-shaped channel extending orthogonally to the axial direction, the u-shaped channel configured to receive the catheter of the medical device.

According to other aspects of the present disclosure, the patency device may include one or more of the following features. The distal end of the occlusive portion may have a domed shape. The distal end may be configured to extend distal to a distal end of the access sheath. The access sheath may be a dual-slit single-access sheath as disclosed herein. The patency device may be comprised of a polymeric material. An outer diameter of the occlusive portion may be less than 5 mm.

According to another aspect of the present disclosure, a kit for thrombus management may include a patency device as disclosed herein and an access or introducer sheath, where the patency device may be configured to be disposed at least partially within a lumen of the access or introducer sheath.

According to another aspect of the present disclosure, a system may include an access or introducer sheath having a lumen extending therethrough. The system may include a patency device as disclosed herein configured to be disposed at least partially within the lumen. The system may include a medical device with a catheter, the catheter disposed within the c-shaped channel of the patency device such that the patency device may be disposed between the catheter and an inner surface of the lumen.

According to other aspects of the present disclosure, the system may include one or more of the following features. The medical device may be a catheter-based intravascular blood pump. The access or introducer sheath may be a dual-slit single-access sheath as disclosed herein. The medical device may be inserted into a first helical slit of the single-access sheath and the patency device may be inserted into a second helical slit of the single-access sheath. The handle of the patency device may be configured to be in contact with a proximal end of the access or introducer sheath.

According to another aspect of the present disclosure, a method may include introducing a medical device through a lumen of an access or introducer sheath, the medical device having a catheter. The method may include passing a portion of catheter through a c-shaped channel in a patency device as disclosed herein. The method may include moving the c-shaped channel distally until the distal end of the patency device passes beyond a distal end of the access or introducer sheath while keeping at least a portion of the intermediate portion of the patency device within the lumen to form a seal.

According to other aspects of the present disclosure, the method may include one or more of the following features. The access or introducer sheath may be a dual-slit single-access sheath as disclosed herein.

According to another aspect of the present disclosure, a patency device for managing thrombus formation may include a tubular member having a proximal end and a distal end, the tubular member having sidewalls defining a lumen extending from the proximal end to the distal end. The patency device may include a linear slit extending radially inward from an outer surface of the tubular member to the lumen, the slit extending axially from the proximal end to the distal end. The lumen may include a proximal portion having a first inner diameter, a distal portion having a second inner diameter different from the first inner diameter, and a transition portion coupling the proximal portion to the distal portion.

According to other aspects of the present disclosure, the patency device may include one or more of the following features. The sidewalls may define a v-shaped cutout extending radially inward from the outer surface to the lumen at the proximal end. The tubular member may be composed of one polymeric material. The transition portion may have a different composition than the distal portion, the proximal portion, or both. The patency device may include a handle at the proximal end, the handle having a main body coupled to the tubular member. The handle may include an opening extending axially from a proximal surface of the handle to a distal surface of the handle, the opening being centered over the lumen. The handle may include at least one tab or protrusion extending radially outward from the main body of the handle. The at least one tab or protrusion may define an opening extending through the at least one tab or protrusion. At least one string, cord, thread, or fiber may be coupled to the opening extending through the at least one tab or protrusion. The main body may extend distally along the outer surface of the tubular member for a portion of the proximal portion of the tubular member. The main body may define one or more opening extending radially outward through the main body. The main body may be a singular component. The main body may include two or more subcomponents coupled to the tubular member. The transition portion may be a tapered region where the inner diameter of the lumen gradually changes from the first inner diameter to the second inner diameter. The transition portion may be a step-change region where the inner diameter of the lumen changes from the first inner diameter to the second inner diameter over one or more step changes in diameter.

According to another aspect of the present disclosure, a system may include an access or introducer sheath having a lumen extending therethrough, and comprising a hemostatic valve. The system may include a patency device as disclosed herein configured to be disposed at least partially within the lumen. At least some of the transition portion including a distal end of the transition portion is axially positioned within the hemostatic valve.

According to other aspects of the present disclosure, the system may include one or more of the following features. The system may include a medical device with a catheter, the catheter extending through the lumen of the patency device.

According to another aspect of the present disclosure, a kit may include a patency device as disclosed herein and an access or introducer sheath, where the patency device may be configured to be disposed at least partially within a lumen of the access or introducer sheath.

According to other aspects of the present disclosure, the kit may include one or more of the following features. The kit may include a loading tool, the loading tool comprising an open channel configured to slidable receive the tubular member of the patency device with the slit of the patency device facing upwards, and two wings coupled to the upper edges of the open channel, the two wings oriented parallel to the open channel and configured to direct a catheter of a medical device into the slit and into the lumen of the patency device.

According to another aspect of the present disclosure, a method may include introducing a medical device through a lumen of an access or introducer sheath, the medical device having a catheter. The method may include passing a portion of catheter through a slit in a patency device as disclosed herein. The method may include moving the patency device distally until the transition portion may be disposed within a hemostatic valve of the access or introducer sheath.

According to other aspects of the present disclosure, the method may include one or more of the following features. The access or introducer sheath may be a dual-slit single-access sheath as disclosed herein. The method may include loading the patency device onto the catheter using a loading tool. The method may include pulling on a handle or a string, cord, thread, or fiber operably coupled to the handle to remove the patency device from the access or introducer sheath.

According to another aspect of the present disclosure, a patency device for managing thrombus formation may include a tubular member having a proximal end and a distal end, the tubular member having sidewalls defining a lumen extending from the proximal end to the distal end. The patency device may include an axially compressible cap at the proximal end of the tubular member, the axially compressible cap defining an opening extending from a proximal surface to a distal surface, the opening centered over the lumen.

According to other aspects of the present disclosure, the patency device may include one or more of the following features. The tubular member may include a linear slit extending radially inward from an outer surface of the tubular member to the lumen, the slit extending axially from the proximal end to the distal end. The axially compressible cap may have a slit extending radially outward from the opening to an outer edge, the slit of the axially compressible cap being aligned with the slit of the tubular member. The sidewalls may define a v-shaped cutout extending radially inward from the outer surface to the lumen at the proximal end. The axially compressible cap may have an outer diameter greater than an outer diameter of the tubular member. The axially compressible cap may include a flexure spring seal. The axially compressible cap may include a polymeric material.

According to another aspect of the present disclosure, a kit may include a patency device as disclosed herein and an access or introducer sheath, where the patency device may be configured to be disposed at least partially within a lumen of the access or introducer sheath, and the axially compressible cap may be configured to be in contact with a proximal end of the access or introducer sheath.

According to another aspect of the present disclosure, a system may include a patency device as disclosed herein and an access or introducer sheath, where the patency device may be disposed at least partially within a lumen of the access or introducer sheath, and the axially compressible cap may be in contact with a proximal end of the access or introducer sheath.

According to other aspects of the present disclosure, the system may include one or more of the following features. The system may include a locking cap, the axially compressible cap being disposed between the proximal end of the access or introducer sheath and the locking cap. The system may include a medical device with a catheter, the catheter extending through the opening in the axially compressible cap and the lumen of the patency device.

According to another aspect of the present disclosure, a method may include inserting a tubular member a patency device as disclosed herein into a lumen of an access or introducer sheath, until the axially compressible cap may be in contact with the proximal end of the access or introducer sheath. The method may include attaching a locking cap to the proximal end of the access or introducer sheath, compressing the axially compressible cap between the access or introducer sheath and the locking cap.

According to other aspects of the present disclosure, the method may include one or more of the following features. The method may include providing a medical device comprising a catheter and inserting the tubular member and axially compressible cap over the catheter.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1A is a depiction of an embodiment of a single-access sheath.

FIG. 1B is a depiction of an embodiment of a hemostatic valve with helical slits.

FIG. 1C is a top-down view of a hub of a single-access sheath.

FIG. 1D is a cut-away side view of a hub of a single access sheath.

FIG. 2A is a depiction of two components of an embodiment of a patency device.

FIGS. 2B-2D are images showing embodiments of a patency device around a catheter.

FIG. 3A is a depiction of an embodiment of a patency device.

FIG. 3B is a close-up depiction of an embodiment of a patency device around a catheter of a medical device, partially within a lumen of a sheath.

FIG. 3C is a depiction of a proximal end of a sheath, showing a catheter passing through a U-shaped channel in a handle of an embodiment of a patency device.

FIG. 4A is a depiction of an embodiment of a patency device.

FIG. 4B is a depiction of an embodiment of a proximal end of a tubular member of a patency device.

FIGS. 4C and 4D are depictions of different handles at a proximal end of a patency device.

FIG. 4E is a schematic depicting placement of an embodiment of a patency device within a sheath.

FIG. 4F is a depiction of an embodiment of a loading tool.

FIG. 5A is a depiction of a proximal end of an embodiment of a patency device.

FIG. 5B is a depiction of an embodiment of a patency device being inserted into a sheath, with a locking cap inserted over it.

FIGS. 5C-5F are images of embodiments of a patency device.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

While intravascular sheaths offer numerous benefits, they can also present challenges related to thrombus formation. When blood comes into contact with foreign surfaces, such as the inner walls of an intravascular sheath, it can trigger the coagulation cascade, potentially leading to the formation of blood clots or thrombi. Thrombus formation within intravascular sheaths poses risks to patient safety, as these clots may dislodge and cause complications such as embolism.

Various approaches have been explored to address the issue of thrombus formation in intravascular sheaths. These include the use of anticoagulant coatings, modifications to sheath materials, and flushing protocols. However, each approach has its limitations, and the management of thrombus risk remains an ongoing concern in interventional procedures.

The duration of sheath placement can influence the likelihood of thrombus formation. Procedures requiring extended sheath dwell times, such as those performed in intensive care settings, may be associated with a higher risk of thrombus development. Additionally, the specific design features of intravascular sheaths, including their internal diameter and surface characteristics, can impact the potential for thrombus formation.

Efforts to mitigate thrombus formation must balance effectiveness with practical considerations such as ease of use, compatibility with existing medical devices, and cost-effectiveness. Furthermore, any solution aimed at reducing thrombus risk should not compromise the primary functions of the intravascular sheath, including maintaining vascular access and hemostasis.

As medical procedures continue to evolve, there is an ongoing need for innovative approaches to enhance the safety and efficacy of intravascular sheaths. Addressing the challenge of thrombus formation could contribute to improved patient outcomes and potentially expand the applications of intravascular procedures.

The disclosed technologies may provide several benefits for managing thrombus formation in intravascular procedures. By utilizing innovative sheath designs and patency devices, these technologies may help reduce the risk of blood clot formation within access sheaths during medical interventions. The dual-slit single-access sheath design may allow for simultaneous insertion of multiple devices through a single access point, potentially reducing procedural complexity and patient discomfort. The various patency devices described may offer improved sealing capabilities and flexibility in positioning within the sheath, which may enhance blood flow management and reduce the likelihood of thrombus formation. Additionally, these technologies may be compatible with existing medical devices and procedures, potentially facilitating their adoption in clinical settings.

The present disclosure encompasses various embodiments of systems, devices, and methods for managing thrombus formation in intravascular procedures. These embodiments may include specialized sheath designs, patency devices with unique sealing mechanisms, and methods for their use. The disclosed technologies may be adapted for use with a wide range of medical devices and may be suitable for various types of intravascular procedures. The embodiments described may be used individually or in combination to address specific clinical needs and may be modified or customized to suit particular applications or patient requirements. The flexibility and adaptability of these technologies may allow for their integration into existing medical workflows and may provide healthcare professionals with additional tools for improving patient outcomes in procedures involving intravascular access.

According to various aspects of the present disclosure, a dual-slit single-access sheath may be provided. Currently, single-access procedures, such as for percutaneous coronary intervention (PCI) procedures, requires puncturing, with a needle at a specific location and angle, a hemostatic valve in an access sheath with a blood pump in place. The puncture must then be dilated using an over-the-wire dilator. Puncturing at an incorrect location or angle may result in damage to the pump or loss of hemostasis. The disclosed dual-slit single-access sheath overcomes these issues.

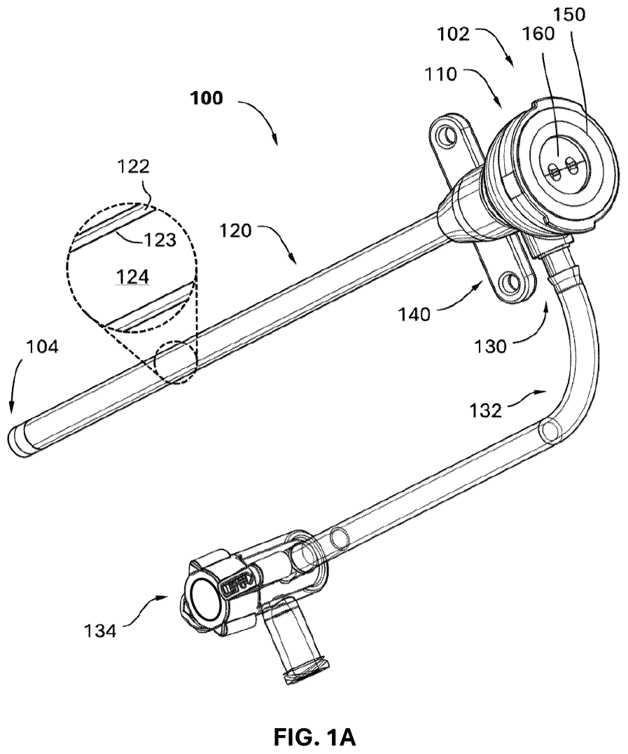

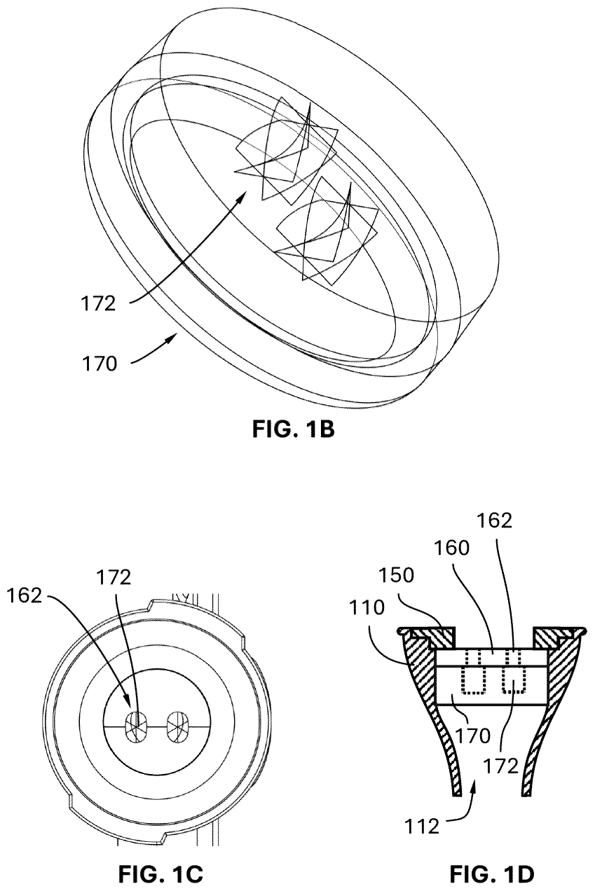

Referring to FIG. 1A, the dual-slit single-access sheath (100) may have a proximal end (102) and a distal end (104). The dual-slit single-access sheath may include a tubular body (120) having sidewalls (122) that define a lumen (124) (with an inner surface (123)) that extends from a proximal end of the tubular member to a distal end of the tubular member. The tubular member may be comprised of any appropriate material. For example, the tubular member may include a polymeric material, such as polyether block amide (such as PEBAX® or PEBASLIX® polyether block amides), a polyethylene material, a polytetrafluoroethylene (PTFE) material, a high-density polyethylene (HDPE) material, a medium-density polyethylene (MDPE) material, and/or a low-density polyethylene (LDPE) material, a silicone polymer, ABS, or a combination thereof. The tubular member may include a metal, such as nitinol, stainless steel, cobalt-chromium alloy, platinum, titanium, tantalum, or a combination thereof. In some embodiments, the metal may be embedded in the sidewalls of the tubular member. A distal end of the tubular body may be blunted (e.g., rounded, chamfered, etc.). a distal end of the tubular body may be tapered.

The sheath (100) may include a hub body (110) operably coupled to a proximal end of the tubular body. The hub body may be any appropriate material, such as a polymeric material. In some embodiments, the polymeric material may be, e.g., a polyether block amide (such as PEBAX® or PEBASLIX® polyether block amides), a polyethylene material, a polytetrafluoroethylene (PTFE) material, a high-density polyethylene (HDPE) material, a medium-density polyethylene (MDPE) material, and/or a low-density polyethylene (LDPE) material, a silicone polymer, ABS, or a combination thereof.

Referring to FIG. 1D, the hub body may define an internal volume of space (112) operably coupled to the lumen (124). The sheath may include a hemostatic valve (170) disposed within the internal volume of space (112). Referring to FIGS. 1B and 1D, the hemostatic valve (170) may include one or two helical slits (172) that extend in an axial direction from a proximal surface towards a distal surface. A cross-sectional shape of each helical slit may vary. In some embodiments, slits are arranged in a regular pattern around a central point. For example, in some embodiments, the slits define six symmetrical arms extending from a central point, each arm rotated 60 degrees from its adjacent arms. The hemostatic valve may be comprised of a polymeric material, such as a silicone polymer.

Referring to FIGS. 1A, 1C, and 1D, the sheath may include a foam pad (160) disposed within the internal volume of space (112), proximal to the hemostatic valve. The foam pad may define one or two openings (162) centered over the one or two helical slits (172). The number of openings matches the number of helical slits. If two openings are used, the two openings may be different-for example, one opening may be larger than the other. In a preferred embodiment, the two openings are identical. The two openings may have a circular cross-section. The two openings may have a non-circular cross-section (e.g., oval, rectangular with rounded ends, etc). A center-to-center spacing of the two openings may be at least 3 mm or at least 4 mm, up to no more than 5 mm, 6 mm, or 7 mm. In preferred embodiments, a center-to-center spacing of the two openings may be 4 mm to 5 mm. The foam pad may be free of additional guiding holes.

The sheath may include an annular cap (150) disposed at least partially within the internal volume, proximal to the foam pad, the central opening of the annular cap configured to allow access to the two openings (162) and the two helical slits (172).

The hub body may include a side port (130) operably coupled to the internal volume of space. The side port (130) may be operably coupled to a valve (134), such as a stopcock valve, via tubing (132).

The sheath may include a suture pad (140) coupled to a distal end of the hub body (110).

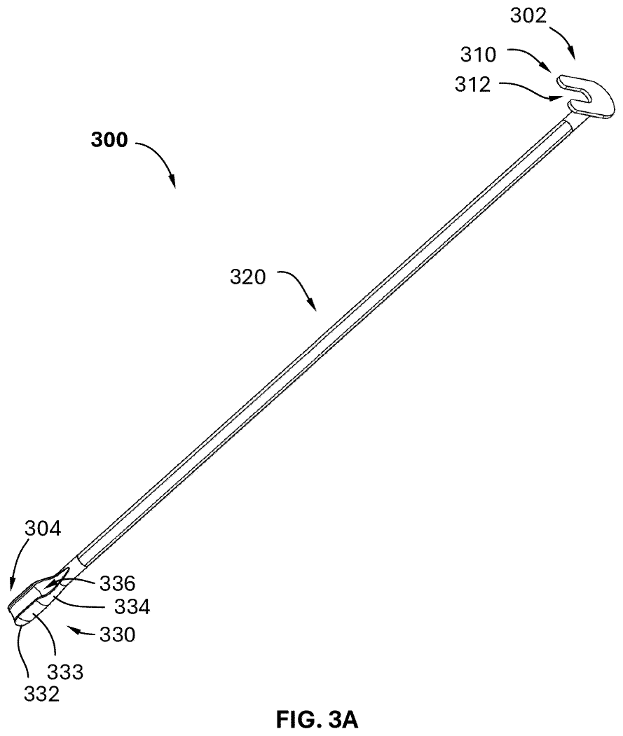

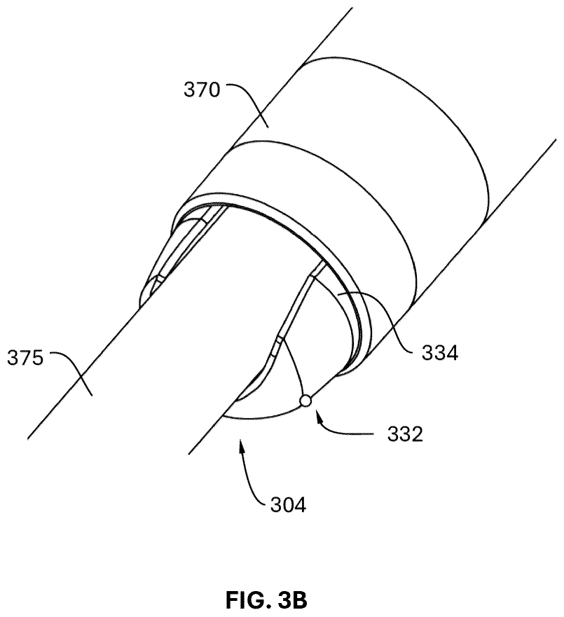

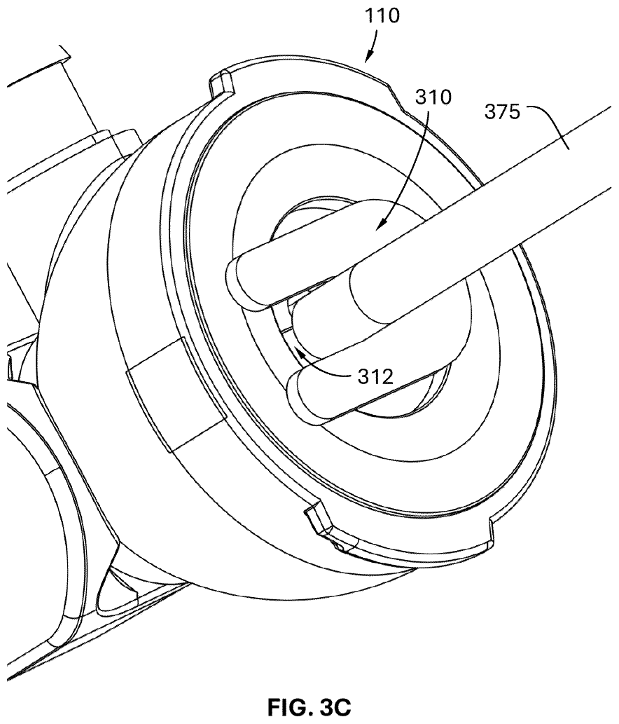

A system may be provided, that includes a dual-slit single-access sheath as disclosed herein and a medical device extending through a first helical slit of the single-access sheath. The medical device may include a catheter. Referring to FIG. 3C, a proximal end of a hub body (110) is shown, where a catheter (375) of a medical device is shown as being inserted through one of the two openings (and one of the two helical slits) of the sheath. A handle (310) of a second medical device (here, a patency device) is also shown inserted through the second opening and helical slit. Alternatively, the system may utilize a percutaneous coronary intervention (PCI) device (such as a balloon catheter, a coronary stent, an imaging device, an atherectomy device, etc.) extending through a second helical slit of the single-access sheath. In some embodiments, the system may include a guide wire extending through the first or second helical slit of the single-access sheath. In some embodiments, the second opening may be kept free of a medical device.

A method of using such sheaths may be provided. The method may include introducing a dual-slit single-access sheath as disclosed herein to a patient via an access point. Any appropriate access point may be used, including a right and/or left femoral artery. The method may include inserting a guide wire through a first helical slit of the single-access sheath and positioning the guide wire within a blood vessel. The method may include inserting a medical device (such as a catheter-based intravascular blood pump) over the guide wire, through the first helical slit, and into a desired position within the blood vessel. The method may include removing the guide wire. The method may include introducing a second guide wire through a second helical slit of the single-access sheath. The method may include introducing a patency device through a second helical slit of the single-access sheath. The method may include introducing a percutaneous coronary intervention (PCI) device through a second helical slit of the single-access sheath.

The sheath will have a lumen with an inner diameter large enough to allow the largest portion of a medical device to pass through the sheath. However, a proximal catheter for the medical devices often has a smaller outer diameter than the inner diameter of the lumen, which leaves gaps that allow blood to flow into the lumen and potentially form a thrombus. Patency devices may utilized to manage or mitigate the risk of thrombus formation, by controlling or preventing blood flow through the lumen.

According to an aspect of the present disclosure, a patency device for managing thrombus formation in a sheath may be provided. Here, a device having a flexible umbrella-like tip and stiffer spine is provided. The device location, blood pressure, and blood flow in a blood vessel can work together to allow the device to seal off the sheath shaft.

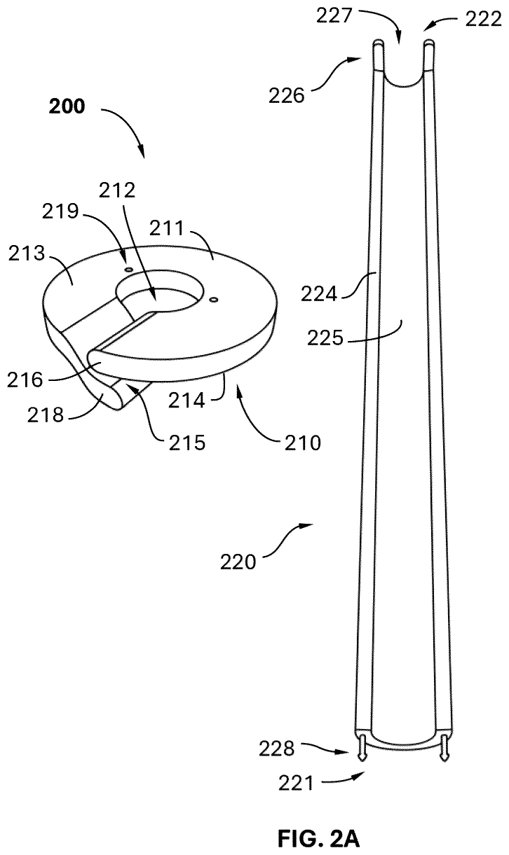

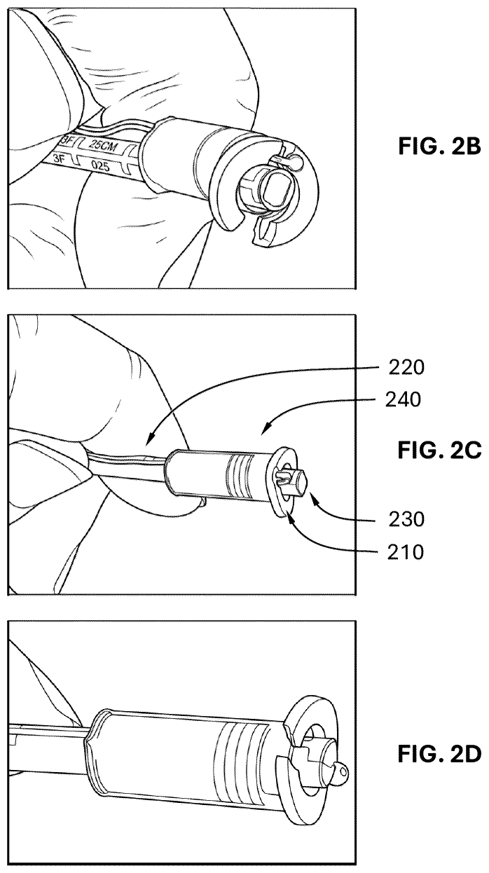

Referring to FIG. 2A, the patency device (200) may include a flexible split ring (210) having a substantially annular body (211) having a split (215) where a first end (216) at least partially overlaps a second end (218). An outer diameter of the flexible split ring may be less than 10 mm. An outer diameter of the flexible split ring may be less than 5 mm.

The flexible split ring (210) may have a central opening (212) extending from a first surface (213) to a second surface (214), the central opening configured to be disposed around a catheter of a first medical device. The central opening may have a diameter of less than 5 mm. The central opening may have a diameter of less than 3 mm.

The flexible split ring may include one or more additional openings (219) extending from the first surface (213) to the second surface (214), each additional opening offset from the central opening.

The patency device may include an elongated body (220) having at least one prong (228) at a first end (221), each prong extending away from the elongated body in an axial direction. Each prong may be configured to extend through one of the additional openings (219), coupling the flexible split ring (210) to the elongated body (220). The patency device may include a handle (226) coupled to the elongated body at a second end (222). The handle may define a U-shaped channel, perpendicular to the axial direction of the elongated body, the U-shaped channel being configured to be disposed at least partially around the catheter of the first medical device.

The elongated body may optionally have sidewalls (224) defining channel (224), such as a c-shaped channel extending from a first end (221) to a second end (222) (e.g., from a distal end to a proximal end, or vice-versa), in which the catheter of a medical device may be slideably received. The channel may be configured to circumferentially surround only a portion of the catheter. The elongate body may have a partial annulus cross-sectional geometry, the partial annulus having a central angle no more than 180 degrees. The elongate body may define a channel extending from a proximal end to a distal end, the channel configured to slideably receive the catheter of the first medical device.

Referring to FIGS. 2B-2D, the elongated body (220) may be configured to be disposed between the catheter (230) of the first medical device and a sheath (240) of a second medical device.

The patency device (200) may be comprised of one or more polymeric materials. For example, the patency device may be comprised of, e.g., a polyether block amide (such as PEBAX® or PEBASLIX® polyether block amides), a polyethylene material, a polytetrafluoroethylene (PTFE) material, a high-density polyethylene (HDPE) material, a medium-density polyethylene (MDPE) material, and/or a low-density polyethylene (LDPE) material, a silicone polymer, ABS, or a combination thereof. The flexible split ring may be comprised of a first polymeric material different from a second polymeric material forming at least part of the elongated body. For example, the flexible split ring may be comprised of LDPE, while the elongated body may be HDPE. Alternatively, the flexible split ring and the elongated body may be comprised of the same polymeric material. The flexible split ring may be comprised of a polymeric material and a metal. The metal may include nitinol, stainless steel, cobalt-chromium alloy, platinum, titanium, tantalum, or a combination thereof.

According to another aspect of the present disclosure, a kit for thrombus management may include a patency device as disclosed herein and an access or introducer sheath (preferably, a dual-slit single-access sheath as disclosed herein), where the patency device may be configured to be disposed at least partially within a lumen of the access or introducer sheath.

According to another aspect of the present disclosure, a system may include an access or introducer sheath having a lumen extending from a proximal end to a distal end. The system may include a patency device as disclosed herein, where the flexible split ring may be in contact with a distal end of the access or introducer sheath and the elongated body extends through the access or introducer sheath. The system may include a medical device (such as an intravascular blood pump) having a catheter, the catheter disposed through the central opening of the flexible split ring, where the elongated body may be disposed between the catheter and an inner surface of the lumen.

According to another aspect of the present disclosure, a method may include introducing a medical device through a lumen of an access or introducer sheath, the medical device having a catheter. The method may include passing a portion of catheter through the slit in a patency device as disclosed herein such that the catheter may be disposed through the central opening of the patency device. The method may include moving the flexible split ring distally until the flexible split ring passes beyond a distal end of the access or introducer sheath, then moving the flexible split ring proximally until a proximal surface of the flexible split ring interacts with the access or introducer sheath and forms a seal. Moving the flexible split ring proximally may include causing a portion of the flexible split ring to be disposed within the lumen. The seal may be a 360 degree seal around the catheter. The seal may be less than a 360 degree seal around the catheter.

Instead of a flexible split ring approach, a patency device may include a rounder obturator that prevents blood ingress into the sheath, using a shaped distal end.

Referring to FIG. 3A, a patency device (300) for managing thrombus formation may include an elongated body (320) having a proximal end (302) and a distal end (304), extending in an axial direction. The elongated body (320) may be free of a lumen extending from the proximal end to the distal end.

The patency device may include an occlusive portion (330) at the distal end (304) of the elongated body, the occlusive portion having a distal end (332), an intermediate portion (333) with a larger diameter than a diameter of the elongated body, and a tapered proximal end (334). An outer diameter of the occlusive portion may be less than 7 mm, 6 mm or 5 mm. An outer diameter of the occlusive portion may be no more than 5 mm, 4 mm, or 3 mm. The intermediate portion (333) may be configured to have a certain length. In some embodiments, the intermediate portion (333) may stop within the lumen it is designed to be placed within. In some embodiments, the intermediate portion (333) may extend to the end of the lumen it is designed to be placed with.

The occlusive portion may define a channel (336), preferably a c-shaped channel, extending in an axial direction. Referring to FIG. 3B, the channel may be configured to wrap partially around a catheter (375) of a medical device, such as a catheter-based intravascular blood pump. The occlusive portion (334) may be configured to be disposed between the catheter (375) and a distal end of a lumen of an access sheath (374), such as the dual-slit single-access sheath disclosed herein.

While the distal end may have any appropriate shape, as seen in FIG. 3B, the distal end of the occlusive portion (i.e., the distal portion (332)) may preferably have a domed shape. Further, the distal end may be configured to stick out from the access sheath, extending distal to a distal end of the access sheath.

Referring to FIG. 3C, the patency device may include a handle (310) having a u-shaped channel (312) that extends orthogonal to the axial direction, the u-shaped channel configured to receive the catheter (375) of the medical device. In some embodiments, the handle has an axial thickness of less than 3 mm, 2 mm, or 1 mm.

The patency device may be comprised of a polymeric material, such as polyether block amide (such as PEBAX® or PEBASLIX® polyether block amides), a polyethylene material, a polytetrafluoroethylene (PTFE) material, a high-density polyethylene (HDPE) material, a medium-density polyethylene (MDPE) material, and/or a low-density polyethylene (LDPE) material, a silicone polymer, ABS, or a combination thereof.

According to another aspect of the present disclosure, a kit for thrombus management may include a patency device as disclosed herein and an access or introducer sheath, where the patency device may be configured to be disposed at least partially within a lumen of the access or introducer sheath.

According to another aspect of the present disclosure, a system may include an access or introducer sheath (such as a dual-slit single-access sheath as disclosed herein) having a lumen extending therethrough. The system may include a patency device as disclosed herein configured to be disposed at least partially within the lumen. The system may include a medical device with a catheter (such as a catheter-based intravascular blood pump). The catheter may be disposed within the channel (336) of the patency device such that the patency device may be disposed between the catheter (375) and an inner surface of the lumen of the sheath (370).

The medical device may be inserted into a first helical slit of the single-access sheath and the patency device may be inserted into a second helical slit of the single-access sheath. The handle of the patency device may be configured to be in contact with a proximal end of the access or introducer sheath.

According to another aspect of the present disclosure, a method may include introducing a medical device through a lumen of an access or introducer sheath (such as a dual-slit single-access sheath as disclosed herein), the medical device having a catheter. The method may include passing a portion of catheter (375) through a channel (376) in a patency device as disclosed herein. The method may include moving the channel (336) distally until the distal end (304) of the patency device passes beyond a distal end of the access or introducer sheath while keeping at least a portion of the intermediate portion (333) of the patency device within the lumen to form a seal (which may be a complete seal, or a partial seal).

Various embodiments of the patency device may provide a seal at a proximal end of the sheath.

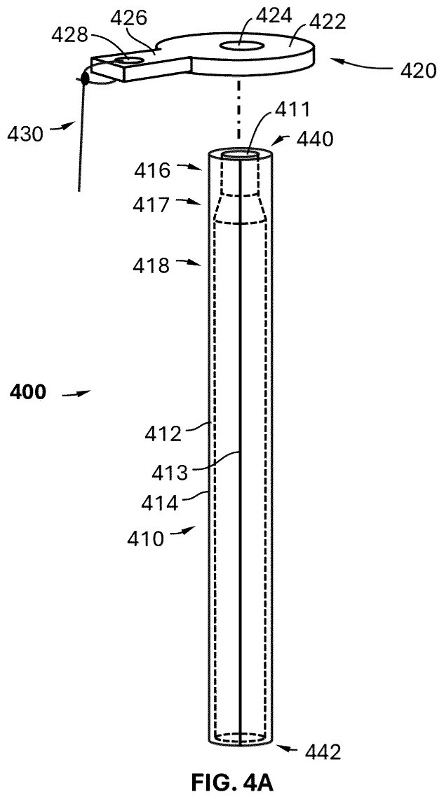

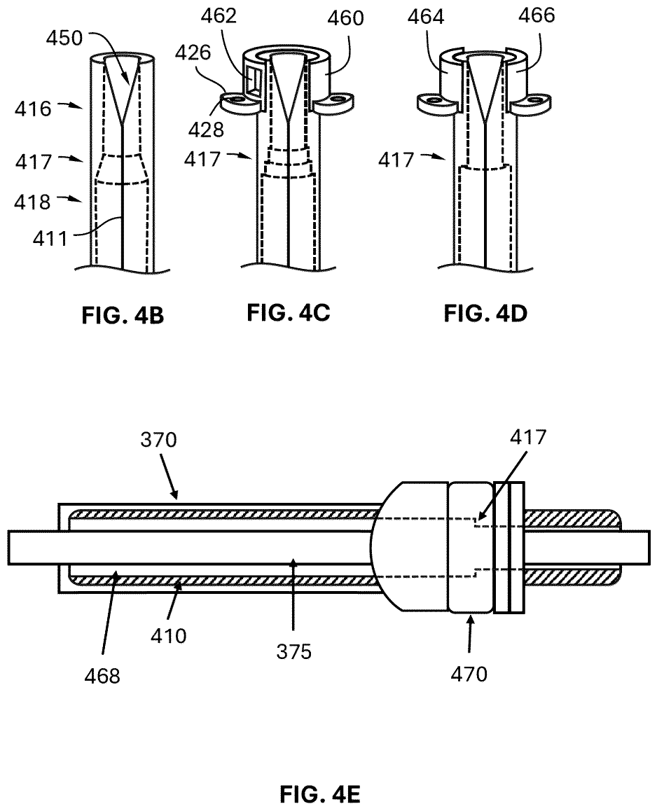

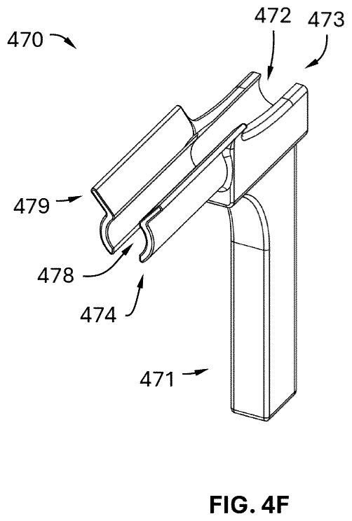

For example, referring to FIG. 4A, a patency device (400) for managing thrombus formation may include a tubular member (410) having a proximal end (440) and a distal end (442). The tubular member may have sidewalls (412) defining a lumen (411) extending from the proximal end to the distal end. The patency device may include a linear slit (413) extending radially inward from an outer surface (414) of the tubular member to the lumen (411). The slit may extend axially from the proximal end to the distal end.

The lumen may two portions with different inner diameters. For example, the lumen may include a proximal portion (416) having a first inner diameter, a distal portion (418) having a second inner diameter different from the first inner diameter, and a transition portion (417) coupling the proximal portion to the distal portion.

As shown in FIGS. 4B-4D, the transition portion may be a tapered region where the inner diameter of the lumen gradually changes from the first inner diameter to the second inner diameter (see FIG. 4B). The transition portion may be a step-change region where the inner diameter of the lumen changes from the first inner diameter to the second inner diameter over one or more step changes in diameter. In FIG. 4C, three steps are used to change from the diameter of the distal portion to the diameter of the proximal portion. In FIG. 4D, a single step is used.

Referring to FIG. 4B, the sidewalls (412) may define a v-shaped cutout (450) extending radially inward from the outer surface to the lumen at the proximal end. As shown in FIG. 4B, the point of the “V” aligns with the slit (411).

The tubular member may be composed of one or more polymeric materials, such as polyether block amide (such as PEBAX® or PEBASLIX® polyether block amides), a polyethylene material, a polytetrafluoroethylene (PTFE) material, a high-density polyethylene (HDPE) material, a medium-density polyethylene (MDPE) material, and/or a low-density polyethylene (LDPE) material, a silicone polymer, ABS, or a combination thereof.

In some embodiments, the tubular member may have a single composition throughout the tubular member. In some embodiments, the transition portion may have a different composition than the distal portion, the proximal portion, or both. For example, the transition portion (417) may be composed of a “softer” polymer material (e.g., having a 30D shore hardness), while the proximal portion (416) and/or distal portions (418) may utilize a “harder” polymer material (e.g., having a 40D shore hardness).

Referring to FIG. 4A, the patency device may include a handle (420) at the proximal end, the handle having a main body (422) coupled to the tubular member. The handle may include an opening (424) extending axially from a proximal surface of the handle to a distal surface of the handle, the opening being centered over the lumen.

The handle may include at least one tab or protrusion (426) extending radially outward from the main body of the handle. The at least one tab or protrusion may define an opening (428) extending through the at least one tab or protrusion. At least one string, cord, thread, or fiber (430) may be coupled to the opening extending through the at least one tab or protrusion. The handle can act as a backstop for the device, so that it cannot be over-inserted into a sheath. Further, the handle and/or cord can provide a grip point for device removal.

While the handle in FIG. 4A is shown as simply coupling to the top surface of the tubular member, the main body of the handle may optionally extend distally along the outer surface of the tubular member for a portion of the proximal portion of the tubular member, as seen in FIGS. 4C and 4D.

The main body may define one or more opening (462) extending radially outward through the main body. The main body may be a singular component (460). The main body may include two or more subcomponents (464, 466) coupled to the tubular member.

According to another aspect of the present disclosure, a system may include an access or introducer sheath (such as a dual-slit single-access sheath as disclosed herein) having a lumen extending therethrough, and comprising a hemostatic valve. The system may include a patency device as disclosed herein configured to be disposed at least partially within the lumen. At least some of the transition portion including a distal end of the transition portion is axially positioned within the hemostatic valve.

The system may include a medical device with a catheter (such as a catheter-based intravascular blood pump), the catheter extending through the lumen of the patency device.

Referring to FIG. 4E, when the transition portion (417), and particularly a portion that includes the distal end of the transition portion is placed under radial compression (such as when the device is inserted through a hemostatic valve (470) of a sheath), the open space (468) between the catheter (375) and the sheath (370) is compressed effectively, preventing blood leaving through the device.

According to another aspect of the present disclosure, a kit may include a patency device as disclosed herein and an access or introducer sheath, where the patency device may be configured to be disposed at least partially within a lumen of the access or introducer sheath.

The kit may include a loading tool. Referring to FIG. 4F, the loading tool may include an open channel (472) configured to slidable receive the tubular member of the patency device with the slit of the patency device facing upwards. The loading tool may have two wings (479) coupled to the upper edges of the open channel, the two wings oriented parallel to the open channel and configured to direct a catheter of a medical device into the slit and into the lumen of the patency device. The open channel may extend from a first end (473) to a second end (474). In some embodiments, a slot (478) may extend from a distal end, proximally along at least a portion of the open channel, as shown in FIG. 4F (the slot being at the bottom of the open channel). As will be appreciated, the slot may extend along any suitable length. In other embodiments, the open channel may not include a slot. The wings may be disposed at the second end. Each wing may extend radially outward from the open channel. The tool may have a handle (471) for ease of use.

According to another aspect of the present disclosure, a method may include introducing a medical device through a lumen of an access or introducer sheath (such as a dual-slit single-access sheath as disclosed herein), the medical device having a catheter (such as an intravascular blood pump). The method may include passing a portion of catheter through a slit in a patency device as disclosed herein. The method may include moving the patency device distally until the transition portion may be disposed within a hemostatic valve of the access or introducer sheath.

The method may include loading the patency device onto the catheter using a loading tool. The method may include pulling on a handle or a string, cord, thread, or fiber operably coupled to the handle to remove the patency device from the access or introducer sheath.

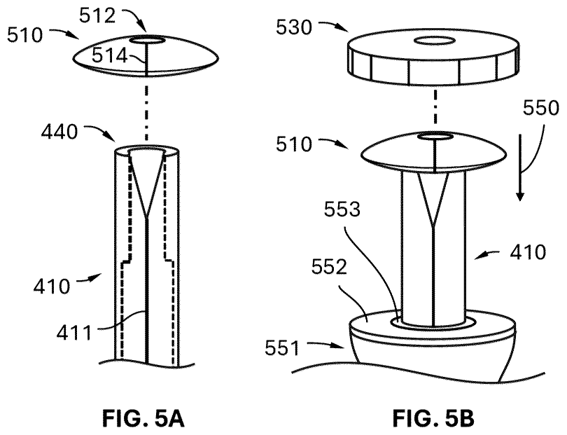

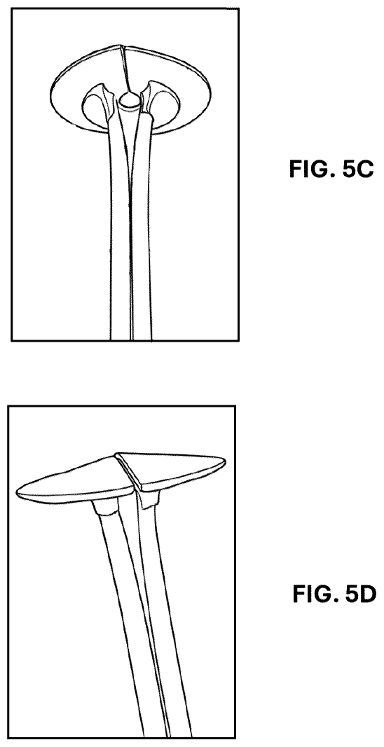

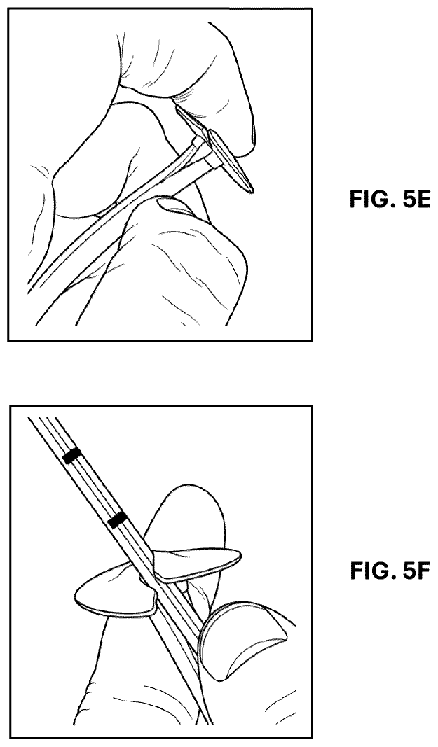

Referring to FIG. 5A, another embodiment of a patency device for managing thrombus formation may include a tubular member (410) having a proximal end and a distal end, the tubular member having sidewalls defining a lumen extending from the proximal end to the distal end. The tubular member may include a linear slit (411) extending radially inward from an outer surface of the tubular member to the lumen, the slit extending axially from the proximal end to the distal end. The sidewalls may define a v-shaped cutout extending radially inward from the outer surface to the lumen at the proximal end.

The patency device may include an axially compressible cap (510) at the proximal end (440) of the tubular member, the axially compressible cap defining an opening (512) extending from a proximal surface to a distal surface, the opening centered over the lumen. The axially compressible cap may have a slit (514) extending radially outward from the opening to an outer edge, the slit (514) of the axially compressible cap being aligned (circumferentially) with the slit (411) of the tubular member. The axially compressible cap may have an outer diameter greater than an outer diameter of the tubular member.

The axially compressible cap may include a flexure spring seal. The axially compressible cap may include a polymeric material. For example, this could include a spring steel (e.g., as ribs or concave discs) with a rubber overmold. This could include a flexible plastic overmold with a silicone rubber secondary molding operation. This could be manufactured via blow molding, or gluing/welding two concave discs together. Thus, the cap may include a polymeric material, such as polyether block amide (such as PEBAX® or PEBASLIX® polyether block amides), a polyethylene material, a polytetrafluoroethylene (PTFE) material, a high-density polyethylene (HDPE) material, a medium-density polyethylene (MDPE) material, and/or a low-density polyethylene (LDPE) material, a silicone polymer, ABS, or a combination thereof. The cap may include a metal, such as nitinol, stainless steel, cobalt-chromium alloy, platinum, titanium, tantalum, or a combination thereof.

According to another aspect of the present disclosure, a kit may include a patency device as disclosed herein and an access or introducer sheath, where the patency device may be configured to be disposed at least partially within a lumen of the access or introducer sheath, and the axially compressible cap may be configured to be in contact with a proximal end of the access or introducer sheath.

A system may include a patency device as disclosed herein and an access or introducer sheath. As shown in FIG. 5B, the patency device may be disposed at least partially within a lumen (553) of the access or introducer sheath (551), and, when fully inserted in the axial direction (550), the axially compressible cap may be in contact with a proximal end (552) of the access or introducer sheath.

The system may include a locking cap (530). When combined, the axially compressible cap may be disposed between the proximal end (552) of the access or introducer sheath (551) and the locking cap (530). The system may include a medical device with a catheter, the catheter extending through the opening in the axially compressible cap and the lumen of the patency device.

A method may include inserting a tubular member a patency device as disclosed herein into a lumen of an access or introducer sheath, until the axially compressible cap may be in contact with the proximal end of the access or introducer sheath. The method may include attaching a locking cap to the proximal end of the access or introducer sheath, compressing the axially compressible cap between the access or introducer sheath and the locking cap.

The method may include providing a medical device comprising a catheter and inserting the tubular member and axially compressible cap over the catheter.

A number of implementations have been described. Nevertheless, it will be understood that various modifications may be made without departing from the spirit and scope of the disclosure. Accordingly, other implementations are within the scope of the following claims.

1-43. (canceled)

44. A patency device for managing thrombus formation, comprising:
an elongated body having a proximal end and a distal end, and extending in an axial direction;
an occlusive portion at the distal end of the elongated body, the occlusive portion having a distal end, an intermediate portion with a larger diameter than a diameter of the elongated body, and a tapered proximal end, the occlusive portion defining a c-shaped channel extending in an axial direction, the c-shaped channel configured to wrap partially around a catheter of a medical device, the occlusive portion configured to be disposed between the catheter and a distal end of a lumen of an access sheath; and
a handle having a u-shaped channel extending orthogonally to the axial direction, the u-shaped channel configured to receive the catheter of the medical device.

45. The patency device of claim 44, wherein the distal end of the occlusive portion has a domed shape.

46. The patency device of claim wherein the distal end is configured to extend distal to a distal end of the access sheath.

47-50. (canceled)

51. A system, comprising:
an access or introducer sheath having a lumen extending therethrough;
a patency device of claim 44 configured to be disposed at least partially within the lumen; and
a medical device with a catheter, the catheter disposed within the c-shaped channel of the patency device such that the patency device is disposed between the catheter and an inner surface of the lumen.

52-57. (canceled)

58. A patency device for managing thrombus formation, comprising:
a tubular member having a proximal end and a distal end, the tubular member having sidewalls defining a lumen extending from the proximal end to the distal end;
a linear slit extending radially inward from an outer surface of the tubular member to the lumen, the linear slit extending axially from the proximal end to the distal end;
wherein the lumen includes a proximal portion having a first inner diameter, a distal portion having a second inner diameter different from the first inner diameter, and a transition portion coupling the proximal portion to the distal portion.

59. (canceled)

60. The patency device of claim 58, wherein the tubular member is composed of one polymeric material.

61. The patency device of claim 58, wherein the transition portion has a different composition than the distal portion, the proximal portion, or both.

62. The patency device of claim 58, further comprising a handle at the proximal end, the handle having a main body coupled to the tubular member.

63. The patency device of claim 62, wherein the handle includes an opening extending axially from a proximal surface of the handle to a distal surface of the handle, the opening being centered over the lumen.

64. The patency device of claim 62, wherein the handle further comprises at least one tab or protrusion extending radially outward from the main body of the handle.

65. The patency device of claim 64, wherein the at least one tab or protrusion defines an opening extending through the at least one tab or protrusion.

66. The patency device of claim 65, wherein at least one string, cord, thread, or fiber is coupled to the opening extending through the at least one tab or protrusion.

67. The patency device of claim 62, wherein the main body extends distally along the outer surface of the tubular member for a portion of the proximal portion of the tubular member.

68. The patency device of claim 62, wherein the main body defines one or more opening extending radially outward through the main body.

69-70. (canceled)

71. The patency device of claim 58, wherein the transition portion is a tapered region where the inner diameter of the lumen gradually changes from the first inner diameter to the second inner diameter.

72. The patency device of claim 58, wherein the transition portion is a step-change region where the inner diameter of the lumen changes from the first inner diameter to the second inner diameter over one or more step changes in diameter.

73-80. (canceled)

81. A patency device for managing thrombus formation, comprising:
a tubular member having a proximal end and a distal end, the tubular member having sidewalls defining a lumen extending from the proximal end to the distal end; and
an axially compressible cap at the proximal end of the tubular member, the axially compressible cap defining an opening extending from a proximal surface to a distal surface, the opening centered over the lumen.

82. The patency device of claim 81, wherein the tubular member further comprises a linear slit extending radially inward from an outer surface of the tubular member to the lumen, the linear slit extending axially from the proximal end to the distal end.

83. The patency device of claim 81, wherein the axially compressible cap has a slit extending radially outward from the opening to an outer edge, the slit of the axially compressible cap being aligned with the slit of the tubular member.

84. The patency device of claim 81, wherein the sidewalls define a v-shaped cutout extending radially inward from an outer surface to the lumen at the proximal end.

85-93. (canceled)
